(19) United States
(12) Patent Application Publication
(10) Pub. No.: US 2021/0374311 A1
(43) Pub. Date: Dec. 2, 2021

(54) SIMULATION TOOL

(71) Applicant: Psylotech, Inc., Evanston, IL (US)

(72) Inventor: G. Alexis Arzoumanidis, Glenview, IL (US)

(21) Appl. No.: 17/341,946

(22) Filed: Jun. 8, 2021

Related U.S. Application Data
(63) Continuation of application No. PCT/US2019/065304, filed on Dec. 9, 2019.
(60) Provisional application No. 62/777,091, filed on Dec. 8, 2018.

(51) Int. Cl. G06F 30/23

(52) U.S. Cl. CPC G06F 30/23; G06F 2111/10

(57) ABSTRACT

A method, stored on a non-transitory medium and executed by a processor, for simulating strain induced orthotropy for a material, comprises calculating three (3) principal strain directions of the simulated material, calculating three (3) distortional strains for the simulated material, and calculating three (3) dilatational strains for the simulated material. The method further comprises calculating free energy for the simulated material, the calculated free energy being calculated from the calculated three principal directions of the simulated material, the three distortional strains and the three dilatational strains. The method yet further comprises calculating, via the calculated free energy, a stress for the simulated material based on the calculated free energy for the simulated material.

### CROSS-REFERENCE TO RELATED APPLICATION

This present application is a continuation of PCT Patent Application Serial No. PCT/US2019/065304, filed on Dec. 9, 2019, entitled “SIMULATION TOOL”, which claims priority to Provisional Application No. 62/777,091, entitled “Simulation Model”, and filed on Dec. 8, 2018, the entirety of which is incorporated by reference herein.

### BACKGROUND OF THE DISCLOSURE

### 1. Field of the Disclosure

The disclosure relates in general to simulation, and more particularly, to viscoelasticity and engineering simulation.

### 2. Background Art

Mathematicians invented the field of mechanics hundreds of years before engineering even existed as an academic discipline. The framework they developed to relate stress to strain is mathematically viable, but nearly devoid of engineering intuition. As a result, complicated, interdisciplinary problems are impractical or even impossible to solve.

### SUMMARY OF THE DISCLOSURE

The disclosure is directed to a method, stored on a non-transitory medium and executed by a processor, for simulating strain induced orthotropy for a material, the method comprising calculating three (3) principal strain directions of the simulated material, calculating three (3) distortional strains for the simulated material, and calculating three (3) dilatational strains for the simulated material. The method further comprises calculating free energy for the simulated material, the calculated free energy being calculated from the calculated three principal directions of the simulated material, the three distortional strains and the three dilatational strains. The method yet further comprises calculating, via the calculated free energy, a stress for the simulated material based on the calculated free energy for the simulated material.

In some configurations, the dilatational energy is defined in terms of large strain according to the following equation:

Δg_{b}=½κ{z₁(ε₁)+z₂(ε₂)+z₃(ε₃)}²,

where z's are dilatation functions and epsilons are in the principal strain directions.

In some configurations, the method further comprises defining the distortional strains as a log of a ratio of stretches of the simulated material according to the following equation:

$${\tilde{γ}}_{3} ≡ {ɛ}_{1} - {ɛ}_{2} = ln ( \frac{{λ}_{1}}{{λ}_{2}} ) ,$$

where \tilde{γ}₃ equals pure shear at small strains, ε₁ and ε₂ are the true strains in principal directions, λ₁ and λ₁ are stretches in perpendicular directions along the simulated material, and defining the distortional strains for the remain faces as a log of a ratio of stretches of the simulated material according to the following equation:

$${\tilde{γ}}_{i} ≡ {ɛ}_{i + 1} - {ɛ}_{i - 1} = ln ( \frac{{λ}_{i + 1}}{{λ}_{i - 1}} ) ,$$

where i=1, 2, 3, 1, 2.

In some configurations, the method further comprises calculated stress is calculated in principal strain directions according to the following equations:

In some configurations, the method further comprises calculating entropic elasticity with a crosslink network in parallel to a generalized Maxell model, the Maxwell elements including nonlinear springs that store energy as volume specific Gibbs free energy, with stress being derived according to the following equation:

$$\frac{\partial Δ {g}_{m}}{\partial {ɛ}_{i}} = {σ}_{i} .$$

The disclosure is also directed to a method, stored on a non-transitory medium and executed by a processor, for simulating stress and strain for an orthotropic composite material, the method comprising calculating six (6) distortional strains for the simulated orthotropic composite material and calculating three (3) dilatational strains for the simulated orthotropic composite material. The method further comprises calculating free energy for the simulated orthotropic composite material, the calculated dilatational energy being calculated from the calculated six distortional strains and the three dilatational strains. The method yet further comprises calculating, via the calculated free energy, a stress for the simulated orthotropic composite material based on the calculated dilatational energy for the orthotropic material.

In some configurations, the dilatational energy is defined in terms of large strain according to the following equation:

Δg_{b}=½κ{z₁(ε₁)+z₂(ε₂)+z₃(ε₃)}²,

where epsilons are the strains in the principal directions of orthotropy, kappa is bulk modulus and the z functions combine into the dilatational contribution to free energy.

In some configurations, the distortional strains are defined by an angle, which leads to a hyperbolic secant function in the stress tensor calculation.

In some configurations, the method further comprises further comprising defining the distortional strains as a log of a ratio of stretches of the simulated material according to the following equation:

$${\tilde{γ}}_{3} ≡ {ɛ}_{1} - {ɛ}_{2} = ln ( \frac{{λ}_{1}}{{λ}_{2}} ) ,$$

where \tilde{γ}₃ equals pure shear at small strains, ε₁ and ε₂ are the true strains in principal directions, and are stretches in perpendicular directions along the simulated material, and defining the distortional strains for the remain faces as a log of a ratio of stretches of the simulated material according to the following equation:

$${\tilde{γ}}_{i} ≡ {ɛ}_{i + 1} - {ɛ}_{i - 1} = ln ( \frac{{λ}_{i + 1}}{{λ}_{i - 1}} ) ,$$

where i=1, 2, 3, 1, 2.

In some configurations, the method further comprises calculating entropic elasticity with a crosslink network in parallel to a generalized Maxell model, the Maxwell elements including nonlinear springs that store energy as volume specific Gibbs free energy, with stress being derived according to the following equation:

$$\frac{\partial Δ {g}_{m}}{\partial {ɛ}_{i}} = {σ}_{i} .$$

In some configurations, the calculated stress is calculated in principal orthotropic directions according to the following equations:

### BRIEF DESCRIPTION OF THE DRAWINGS

The disclosure will now be described with reference to the drawings wherein:

FIG. 1 of the drawings illustrates an example simulation system, a version of which may comprise a control module, in accordance with the embodiments disclosed herein;

FIG. 2A illustrates an example of 3 diamonds on a cube deforming in tension, in accordance with the embodiments disclosed herein;

FIG. 2B illustrates an example traditional shear on 3 planes, dubbed 4,5,6, in accordance with the embodiments disclosed herein;

FIG. 2C illustrates an example of volume change from 3 orthogonal strains, in accordance with the embodiments disclosed herein;

FIG. 3 illustrates an example shape of Gibbs Free Energy curves vs. shear strain and the resulting stress-strain relationship, in accordance with the embodiments disclosed herein;

FIG. 4 illustrates an example of Morse potential energy function used to inform Bulk Free Energy function, such as the illustrated Morse potential energy function vs. strain, in accordance with the embodiments disclosed herein;

FIG. 5 illustrates an example generalized Maxwell model, applicable to bulk or shear. Gibbs Free Energy change can be tracked by monitoring strain in each spring shown, in accordance with the embodiments disclosed herein;

FIG. 6 illustrates an exemplary general-purpose computing device, in accordance with the embodiments disclosed herein;

FIG. 7 illustrates an example simulation system performing an example simulation of a material sample that is subject to stretches λ₁ and λ₂ in perpendicular directions along the simulated material, in accordance with the embodiments disclosed herein;

FIGS. 8A and 8B illustrate a first example graph including distortional A-strain as the x-axis and two curves, an energy curve and a shear stress curve, and a second example graph including 1D strain as the x-axis and three curves, a z(ε) strain curve, a Morse energy curve, and a Morse stress curve, respectively, in accordance with the embodiments disclosed herein;

FIG. 9 illustrates example calculation of distortion in 6 axis and dilation in 3 axis, in accordance with the embodiments disclosed herein;

FIGS. 10A and 10B illustrate a first example graph including distortional A-strain as the x-axis and two curves, an energy curve and a shear stress curve, in accordance with the embodiments disclosed herein;

FIG. 11 illustrates a second example graph including 1D strain as the x-axis and three curves, a z(ε) strain curve, a Morse energy curve, and a Morse stress curve, in accordance with the embodiments disclosed herein;

FIG. 12 illustrate an example method 1200 for simulating strain induced orthotropy for a material, in accordance with the embodiments disclosed herein; and

FIG. 13 illustrates an example method for simulating stress and strain for an orthotropic composite material, in accordance with the embodiments disclosed herein.

### DETAILED DESCRIPTION OF THE DISCLOSURE

While this disclosure is susceptible of embodiment in many different forms, there is shown in the drawings and described herein in detail a specific embodiment(s) with the understanding that the present disclosure is to be considered as an exemplification and is not intended to be limited to the embodiment(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

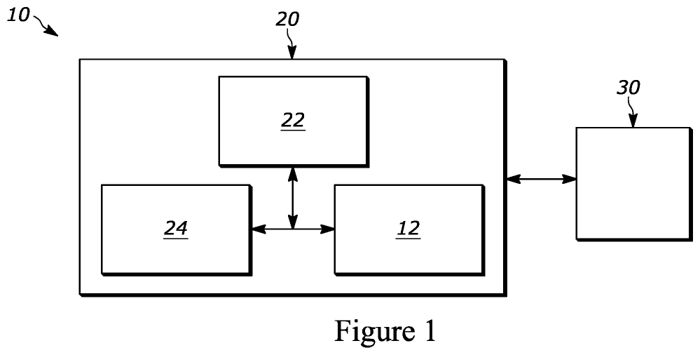

Referring now to the drawings and in particular to FIG. 1, a simulation system 10 is disclosed herein, linking materials science, thermodynamics, mechanics and failure into a single process. The simulation system 10 includes a simulation module 12, a communication module 22, and a programming module 24, each being coupled to each other as shown. The simulation system 10 uses these new mechanics to make previously difficult problems manageable for the example disciplines:

Nonlinear Viscoelasticity—plastic & rubber material properties change with loading history, temperature, and environment. The simulation system 10 simulates these viscoelastic materials. Alternate viscoelastic constitutive models can cover some narrow range of loading & environmental conditions. The simulation system 10 disclosed herein can handle any 3D loading & any temperature history.

Viscoelastic Adhesive Bond Fracture—modern fracture mechanics cannot describe time and temperature dependent crack growth in polymeric adhesive bonds. The simulation system 10 disclosed herein can unify mechanics & fracture into a single process, enabling a solution.

Composites—unlike the current state of the art, the simulation system 10 disclosed herein can simulate glass and carbon fiber composites on a continuum level, thereby accelerating computation time and also provide for tracking of viscoelastic damage accumulation.

Foams—closed cell polymeric foams in particular are difficult to simulate. The simulation system 10 accounts for the pneumatic, localization and microstructural effects that complicate modeling these materials.

Molecular Dynamics Scaleup—quantum mechanics are used to simulate new material chemistries on a nanometer scale. The simulation system 10 provides an unprecedented path to scale these nanometer results to a macro scale.

Non-Newtonian Fluids—the simulation system 10 uses a unified theory that also applies to viscoelastic fluids, with substantial implications for tribology and polymer processing.

Plasticity—the study of permanent deformation in metals is called plasticity. The simulation system 10 clarifies shear yield criteria and seamlessly integrates failure by cavitation.

Bi-Axial Testing—the new mechanics disclosed herein eliminate the need for complicated, expensive and often inaccurate bi-axial experiments.

Shock Physics—the new mechanics disclosed herein simplify shape charge applications, as used in armor or oil industry casing perforation applications.

The simulation system 10 uses a new mathematical framework as part of engineering simulations, which are used in engineering product design. Examples of numeric simulation include finite element analysis, finite difference, and multi-body simulations.

The simulation system 10 combines four siloed engineering subjects into a single process: Materials Science, Thermodynamics, Mechanics and Fracture/Failure/Plasticity. The simulation system 10 shifts focus in mechanics from stress-strain to free energy-strain relationships, revealing a unified theory for solid, fluid and viscoelastic mechanics. The disclosure starts with the relationship between thermodynamics and solid mechanics, then integrates viscoelasticity concepts, which in turn can be applied to fluid mechanics.

Mathematicians gave engineers solid mechanics that relate stresses to strains. The simulation system's 10 unified approach relates free energy change to strains instead. Stresses are then calculated as the derivative of free energy with respect to each of the strains in accordance with the following formula:

$${σ}_{i} = \frac{\partial Δ {g}_{m}}{\partial {ɛ}_{i}} \qquad ( 1 )$$

where σᵢ are the 6 elements in the stress tensor, εᵢ are the 6 elements in the strain tensor and, Δgₘ is the change in Gibbs Free Energy from mechanical deformation. Equation (1) is important in that it is the bridge between thermodynamics and mechanics. Traditional mechanics typically relate stresses to strains directly.

The simulation system 10 further includes the programming module 24. The programming module 24 comprises a user interface which can configure the simulation system 10. In many instances, the programming module 24 comprises a keypad with a display that is connected through a wired connection with the control module 20. Of course, with the different communication protocols associated with the communication module 22, the programming module 24 may comprise a wireless device that communicates with the control module 20 through a wireless communication protocol (i.e., Bluetooth, RF, WIFI, etc.). In other embodiments, the programming module 24 may comprise a virtual programming module in the form of software that is on, for example, a personal computer, in communication with the communication module 22. In still other embodiments, such a virtual programming module may be located in the cloud (or web based), with access thereto through any number of different computing devices. Advantageously, with such a configuration, a user may be able to communicate with the simulation system 10 remotely, with the ability to change functionality.

In at least one embodiment, the simulator system 10 is coupled to a manufacturing system 30. The manufacturing system 30 receives the simulation results produced by the simulation system 10 and manufactures one or more physical products from the simulation results produced by the simulation system 10. For example, the manufacturing system 30 can manufacture any of the example products discussed herein, although other physical products are also contemplated.

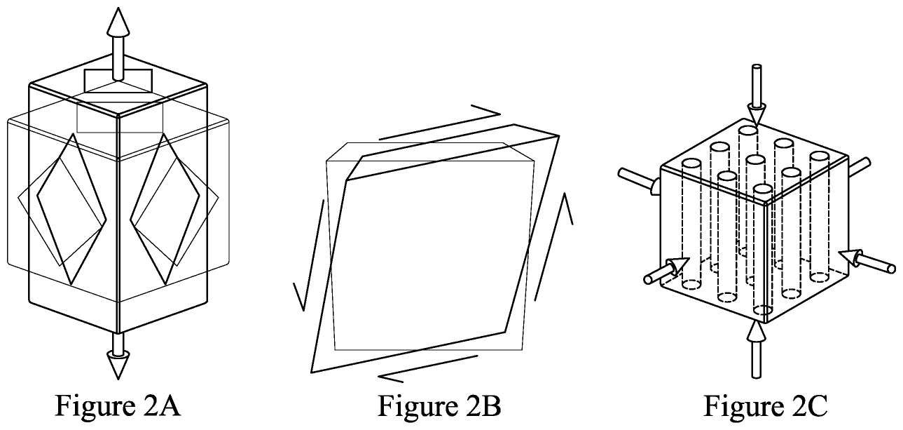

Energy methods have been implemented over the centuries, but the present simulation system 10 introduces a crucial difference. The energy function is separated into 6 independent shears (distortion) and 3 interrelated functions defining bulk (dilatation). To visualize this, FIGS. 2A-2C illustrate cube elements representing deformation. In particular, FIG. 2A illustrates 3 diamonds on a cube deforming in tension, dubbed shear 1,2,3, FIG. 2B illustrates traditional shear on 3 planes, dubbed 4,5,6, and FIG. 2C illustrates volume change from 3 orthogonal strains.

FIG. 2A depicts tension on a cube. In the undeformed state, the diamond shapes start as squares 45 degrees on each face. If the simulated material is initially isotropic (same properties in all 3 directions), tension causes the diamonds on two faces to distort into rhombuses and the square on the face perpendicular to loading remains a square, experiencing dilatation but zero distortion.

FIG. 2B represents one of the 3 traditional shear strains. These shears are common to the embodiments disclosed herein and traditional mechanics. By contrast, FIG. 2C represents something completely unique compared to tradition. This is one of the keys to the simulation system 10. As an example, for a uniaxial composite simulated material, hydrostatic pressure causes different strains in the three different directions. The simulation system 10 assigns different material properties to volume change from each of the three different directions. Typically, volume change is related to pressure by a single property: bulk modulus.

In the most general case, a free energy function incorporates these 6 shear and 3 bulk relationships as follows:

Δgₘ=⅔Σᵢ₌₁³Δgᵢ(γᵢ)+Σᵢ₌₄⁶Δgᵢ(γᵢ)+Δg_{B}(ε₁,ε₂,ε₃) (2)

where Δgᵢ(γᵢ) is the change in the volume-specific Gibbs Free Energy, which is a function of only the i'th shear strain. The 6 energy vs. shear relationships are independent of each other. The change in Gibbs Free Energy from volume change is a function of the logarithmic strains in the 3 directions, Δg_{B}(ε₁, ε₂, ε₃). Changes in volume can influence the 6 shear responses, particularly for simulated viscoelastic materials.

Combining Equations (2) and (1) results in 6 stress-strain relationships:

$${σ}_{1} = \frac{2}{3} sech ( {ɛ}_{3} - {ɛ}_{2} ) ( \frac{\partial Δ {g}_{3}}{\partial {γ}_{3}} - \frac{\partial Δ {g}_{2}}{\partial {γ}_{2}} ) + \frac{\partial Δ {g}_{B}}{\partial {ɛ}_{1}} \\ {σ}_{2} = \frac{2}{3} sech ( {ɛ}_{1} - {ɛ}_{3} ) ( \frac{\partial Δ {g}_{1}}{\partial {γ}_{1}} - \frac{\partial Δ {g}_{3}}{\partial {γ}_{3}} ) + \frac{\partial Δ {g}_{B}}{\partial {ɛ}_{2}} \\ {σ}_{3} = \frac{2}{3} sech ( {ɛ}_{2} - {ɛ}_{1} ) ( \frac{\partial Δ {g}_{2}}{\partial {γ}_{2}} - \frac{\partial Δ {g}_{1}}{\partial {γ}_{1}} ) + \frac{\partial Δ {g}_{B}}{\partial {ɛ}_{3}} \\ {σ}_{4} = \frac{\partial Δ {g}_{4}}{\partial {γ}_{4}} \\ {σ}_{5} = \frac{\partial Δ {g}_{5}}{\partial {γ}_{5}} \\ {σ}_{6} = \frac{\partial Δ {g}_{6}}{\partial {γ}_{6}} \qquad ( 3 )$$

For example, consider simulated orthotropic materials, which have unique properties in the 3 orthogonal directions. Continuous fiber composites can be orthotropic. For the limited case of small strain, linear elastic orthotropy, all the energy functions are essentially parabolas:

Δgₘ=⅔Σᵢ₌₁³½μᵢγᵢ²+Σᵢ₌₄⁶½μᵢγᵢ²+½Σᵢ₌₁³(εᵢ\sqrt{κᵢ})² (4)

where μᵢ are 6 shear moduli, γᵢ are the 6 shear strains, εᵢ are logarithmic strains and κᵢ are 3 properties related to volume change. Combining (1) and (4) results in the orthotropic linear elastic stiffness tensor:

$$\{ \begin{matrix}{σ}_{11} \\ {σ}_{22} \\ {σ}_{33}\end{matrix} \} = [ \begin{matrix}{κ}_{1} + \frac{2}{3} ( {μ}_{2} + {μ}_{3} ) & \sqrt{{κ}_{1} {κ}_{2}} - \frac{2}{3} {μ}_{3} & \sqrt{{κ}_{1} {κ}_{3}} - \frac{2}{3} {μ}_{2} \\ \sqrt{{κ}_{2} {κ}_{1}} - \frac{2}{3} {μ}_{3} & {κ}_{2} + \frac{2}{3} ( {μ}_{1} + {μ}_{3} ) & \sqrt{{κ}_{2} {κ}_{3}} - \frac{2}{3} {μ}_{1} \\ \sqrt{{κ}_{3} {κ}_{1}} - \frac{2}{3} {μ}_{2} & \sqrt{{κ}_{3} {κ}_{2}} - \frac{2}{3} {μ}_{1} & {κ}_{3} + \frac{2}{3} ( {μ}_{2} + {μ}_{1} )\end{matrix} ] \{ \begin{matrix}{ɛ}_{11} \\ {ɛ}_{22} \\ {ɛ}_{33}\end{matrix} \} \qquad ( 5 )$$

Textbooks describing mechanics of composites recognize orthotropic materials need 9 independent properties, but they normally use 3 Young's moduli, 3 Poisson's ratios and 3 shears. Equation (5) describes the upper left-hand quadrant of the stiffness tensor in a form such that the 9 orthotropic properties are 6 shear and 3 bulk.

But Equation (5) is only valid for the small strain, linear elastic response. Equations (2) and (3) are substantially more powerful, as they are valid for all strains. A question becomes what are the shapes of these energy functions? Consider first the 6 shear energy relationships in initially orthotropic materials. The shear stress-strain relationships must meet 4 requirements.

1. Nearly linear at small strains, as defined by shear modulus;

2. Antisymmetric, so the response is identical for positive or negative shear strain;

3. An instability (local maximum/minimum) to trigger crack growth or plasticity; and

4. Failure must eventually occur, as shear stress must be zero at some non-zero strain.

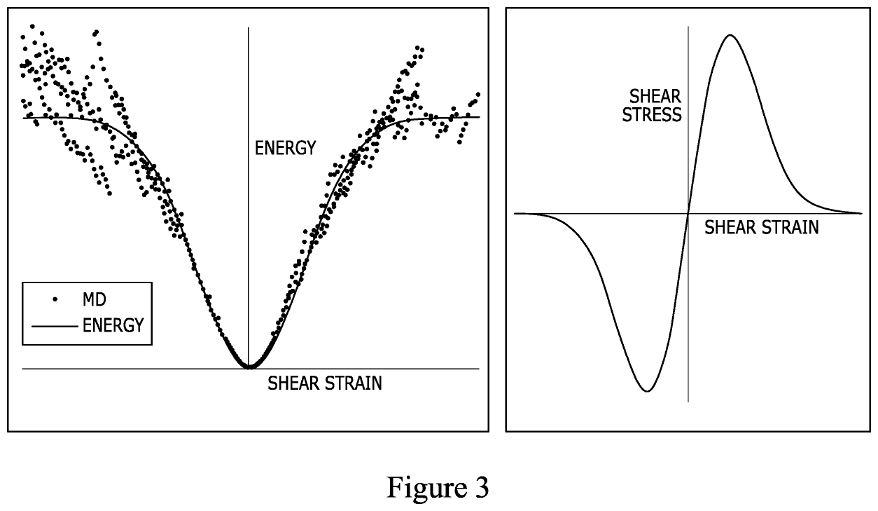

FIG. 3 illustrates shape of Free Energy curves vs. shear strain and the resulting stress-strain relationship. An inverted Gaussian distribution for the Gibbs function satisfies these requirements, as illustrated in FIG. 3. Note, the energy function could take any shape, as motivated by understanding of materials science, micromechanics, nanomechanics, and/or molecular dynamics (MD) simulation. This is how materials science is tied to thermodynamics, as disclosed herein.

For bulk modulus, first consider the 1D Morse Potential Energy function well known in Materials Science. Converted to strain, this function takes on the form:

$$V = \frac{E}{2} {( \frac{1 - {e}^{- c ɛ}}{c} )}^{2} \qquad ( 6 )$$

where V is potential energy, E is Young's modulus, ε is 1D engineering strain, and c is a defining parameter.

Simulated materials can get more complicated than orthotropic (different properties in the 3 orthogonal directions). For example, simulated materials can be anisotropic or monoclinic. Also, microstructure can influence response, such a simulation result produced by the simulation system 10. Take for example pulling on a rope. Axial force can cause torsion as the rope tries to unwind. In all these cases, the Gibbs Free Energy function can include extra terms to address these types of simulated materials.

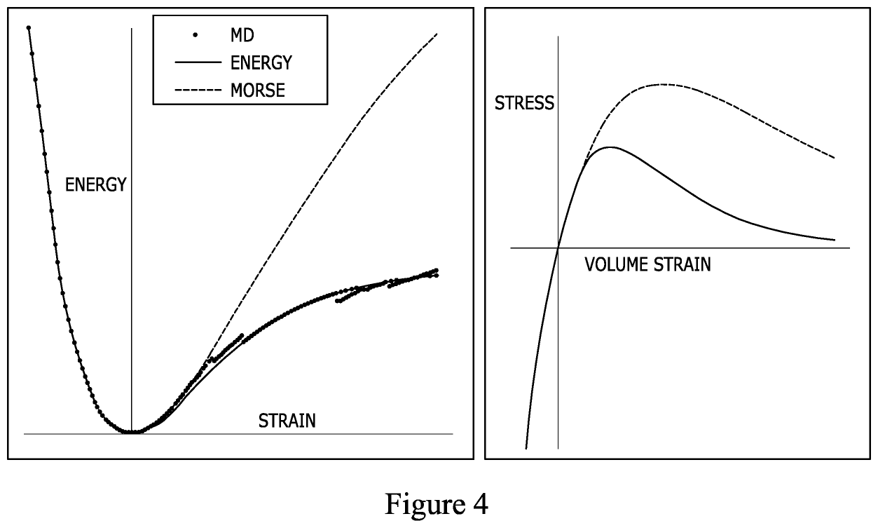

FIG. 4 illustrates Morse potential energy function used to inform Bulk Free Energy function, such as the illustrated Morse potential energy function vs. strain. The Morse Potential balances the repulsive and attractive atomic forces keeping two atoms together. As the atoms are pushed close, repulsion dominates, and the simulated material becomes extremely stiff. In tension, the atoms eventually lose attraction and their connection ultimately fails.

The simulation system 10 may use 1D Morse to motivate the 3D bulk Free Energy function for initially isotropic polymers:

$$Δ {g}_{B} = \frac{κ}{2} {( \frac{1 - {e}^{- c {ɛ}_{1}}}{c} + \frac{1 - {e}^{- c {ɛ}_{2}}}{c} + \frac{1 - {e}^{- c {ɛ}_{3}}}{c} )}^{2} \qquad ( 7 )$$

Equation (7) is the first step of building a bulk energy function for a given simulated material and is meant to be an example of the process. Like shear, the bulk free energy function can also be motivated by materials science, micromechanics, nanomechanics or molecular dynamic simulation.

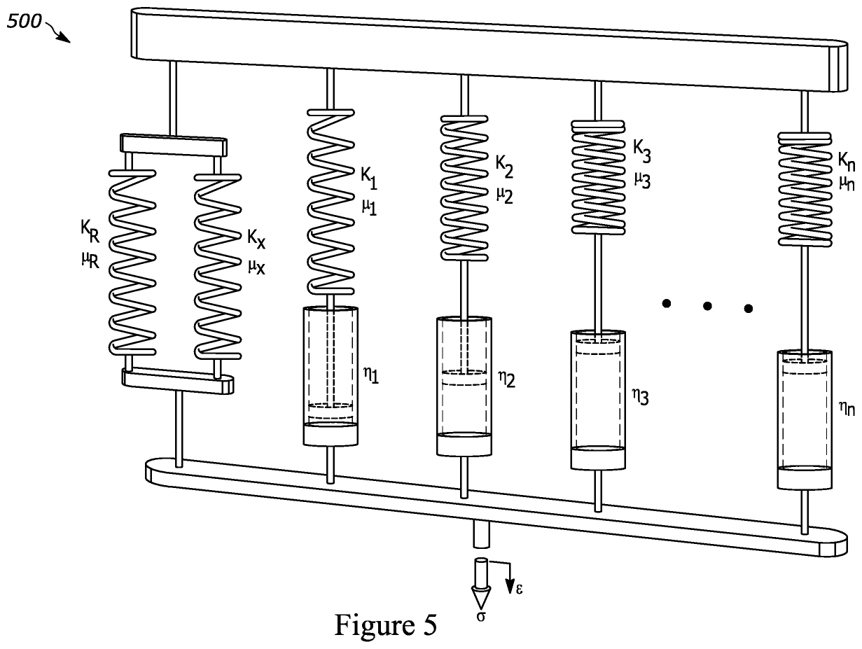

The description thus far describes solid mechanics, which means no time dependence. A simulated material's mechanical response can also have a viscous contribution. A mechanical analog is a helpful tool for understanding the so-called viscoelastic response. Consider the generalized Maxwell model mechanical analog illustrated in FIG. 5, as utilized by the simulation system 10. FIG. 5 illustrates a generalized Maxwell model 500, applicable to bulk or shear. Gibbs Free Energy change can be tracked by monitoring strain in each spring shown.

The Gibbs free energy functions in the new mechanics of the simulation system 10 are applied to the springs in the FIG. 5. Time dependence then comes from the viscosity in the dashpots. Each spring-dashpot pair is known as a Maxwell element, and the Maxwell elements can be combined in parallel to produce a discretized spectral response, representing stiffness as a function of time. For example, each spring-dashpot pair can fit a simulated material's time dependent master curve or describe a simulated material's frequency response. Note, also, other functions besides Prony Series and Maxwell Elements could be used to capture time dependent modulus.

Reduced Time Models are a class of viscoelastic constitutive models used to model plastics, rubbers, and glasses. In reduced time, the time dependence is accelerated by loading history and environment. For example, in time-temperature superposition, raising temperature accelerates the time dependent response. The simulation system 10 accommodates all environmental conditions and mechanical loading histories:
Raising temperature accelerates time;
Increasing volume in mechanical loading accelerates time for the shear response;
Increasing free energy for each Maxwell element accelerates time for that element;
Entropic elasticity in rubber decelerates time for all Maxwell elements; and
Solvent absorption accelerates time.

In a Tabular Format:

| | Accelerates | Decelerates |
|---|---|---|
| Temperature | [symbol] | |
| Volume | [symbol] | |
| Solvents | [symbol] | |
| Entropic Elasticity | | [symbol] |
| Maxell element-specific Free Energy | [symbol] | |

An aspect of the simulation system 10 is implementing nonlinear springs in the Maxwell elements. This innovation enables viscoelastic damage tracking as well as time & temperature dependent fracture.

The simulation system 10 is sufficiently general to cover fluid mechanics, including viscoelastic fluids. To do so, the simulation system 10 simply removes the twin springs on the left of FIG. 5. The resulting unifying theory explains all non-Newtonian fluids: Bingham, shear thinning, and shear thickening. Just like viscoelastic solids, the theory incorporates pressure effects on fluid viscosity, which is very significant for tribology simulation and polymer processing.

Thus, in accordance with the disclosure the simulation system 10 implements numeric simulations based on 6 shear strains and 3 axial strains. The approach disclosed herein contrasts traditional 3 axial and 3 shear strain tensor and is valid for solids, fluids or viscoelastic materials. Small strain, orthotropic, linear elastic stiffness tensor built on 3 bulk and 6 shear moduli are possible in accordance with this disclosure. Note, this reduces to one bulk and one shear for small strain, linear isotropy. Numeric simulations can be implemented based on six independent shear free energy-strain relationships and one bulk free energy-strain relationship. The bulk relationship is based on 3 orthogonal logarithmic strains. The approach disclosed herein contrasts the direct stress-strain approach and is valid to large strains. Each shear free energy-strain relationship can be a function, some combination of functions, or a spline fit. It meets 4 criteria:

1. Symmetric around zero strain (see FIG. 3);
2. Zero slope at large strain (i.e., failure at large strain);
3. Nearly parabolic at small strain; and
4. Derivative has a peak to drive failure instability.

The simulation system 10 can implement upside-down Gaussian distribution for shear energy for mathematical convenience in accordance with this disclosure. An aspect of the simulation system 10 disclosed herein is the form of the bulk modulus energy relationship:

Δg_{B}=½(Σᵢ₌₁³zᵢ\sqrt{κᵢ})² (8)

where zᵢ is some function of εᵢ. This could be a function, a patchwork of functions covering smaller ranges, or a cubic spline. For example, to generate the Morse Potential inspirited function in Equation (7),

$${z}_{i} = \frac{1 - {e}^{- c {ɛ}_{i}}}{c} , i = 1 , 2 , 3 \qquad ( 9 )$$

Accelerated Computation Time: traditional nonlinear solvers must concurrently minimize 6 constitutive relationships. Since the 6 strains are independent of each other, these 6 nonlinear equations are minimized one at a time, which is faster. Nonlinear bulk would still require 3 concurrent minimizations. The simulation system 10 can implement 6 shear and 3 bulk strains in Digital Image Correlation (DIC). Logarithmic strains are already reported in such DIC software, such as ε₁₁, ε₂₂ and γ₁₂. The simulation system 10 can report ε₁, ε₂, γ₃, and γ₄, as per FIG. 2.

For nonlinear viscoelasticity, implementing the simulation system 10 disclosed herein results in a reduced time constitutive model for plastic & rubber. A key contribution of the simulation system 10 is using thermodynamic sub-states to accelerate time. To help understand this, consider FIG. 5. Rather than using free energy of a simulated system to accelerate time, each individual Maxwell element has its own Gibbs free energy state. Thermal shifting factors are measured directly, and curve fit with a spline. This approach eliminates the need for thermorheologic simplicity. Volume change affects viscoelastic shear moduli, even though the 6 shear relationships are independent of each other. This is related to pressure decelerating time for viscoelastic shear. Two springs are placed in parallel with Maxwell elements, such as shown in FIG. 5, one spring for entropic elasticity and the other spring representing the hyperelastic cross-link network. Entropic elasticity decelerates time for all 6 viscoelastic spectral shear relationships. This aspect of the simulation system 10 is key for rubber simulation. Vertical shifting: the entropic elastic spring stiffens with increasing temperature and all other springs soften with temperature, as shown in FIG. 5.

A parallel hyperelastic spring shown in FIG. 5 can be a modified Gent/Arruda-Boyce:

1. Different crosslink network stiffening parameter in uniaxial tension vs. compression;
2. For 3D loading, use in-plane area to modify stiffening parameter;
3. Multiplied by an exponential function to provide peak in stress-strain (failure);
4. Includes a foam-like localization parameter; and
5. Mullins tracked through shifting network stiffening parameter in shear.

Change in entropy state plus irreversible entropy generated by dash pots informs heat generated by mechanical loading. The resulting heat changes temperature, which can feed back into the mechanical loading and change response.

Include viscoelastic bulk response in model. Alternative models assume incompressibility.

For fracture & failure criteria, the 6 shear and 3 bulk stress-strain curves have a peak. This peak represents an instability, which can be used to mathematically trigger crack growth. Nonlinear springs in the generalized Maxwell model of FIG. 5 can solve time & temperature dependent, mixed-mode adhesive bond fracture. Adding a damage state variable to each Maxwell element (MWE) can enable viscoelastic damage buildup in fatigue fracture. As each MWE reaches the peak in the spring's stress-strain response, its ability to hit that peak would be degraded. Unlike the status quo, failure can also be either distortional or dilatational. Degradation could change peak strain, but still revert to initial zero strain at zero stress. This is a way bulk damage could respond, though bulk compression is impervious to damage built-up. Degradation could change initial strain at zero stress, leading to permanent deformation. This is a plausible case for shear. Note, shear requires twice the damage internal state variables, one for positive and one for negative strain, relating free energy to strain.

Strain energy density is a cornerstone of traditional fracture mechanics. Strain energy release rate (G) is the fracture prediction material property. The famous J-integral determines energy at the crack tip through a surface integral measuring energy of the structure around the crack. But traditional fracture mechanics comes from traditional mechanics, which relates 6 stresses to 6 strains. The simulation system 10 combines solid mechanics and fracture mechanics into a unified mechanics.

The simulation system 10 can replace cohesive zone models, which are another fracture simulation approach in finite element analysis, used to predict Mixed-Mode fracture, particularly for adhesive bonds. This approach defines traction separation (TS) laws for Mode I (opening) and Mode II (shear) crack growth. In the current state of the art, TS laws cannot capture rate, time or temperature dependence. The simulation system 10 integrates TS-type laws into the nonlinear springs, using distortion & dilatation instead of Modes I and II. The simulation system 10 therefore naturally accommodates time/temperature effects on polymer adhesive bonds.

Fatigue fracture models also exist, notably as implemented by Endurica. These models presume cracks open only in Mode I, do not properly track heat build-up from mechanical cycling, and typically ignore temperature & rate effects on viscoelastic material properties.

For composites, the simulation system 10 provides for viscoelastic dilatational damage buildup in polymer matrix composites, viscoelastic distortional damage buildup in polymer matrix composites, and applies the 9 properties of 6 shear and 3 bulk to orthotropic, composite materials on the continuum level.

Linear elastic orthotropic materials are known to require 9 independent material properties. Traditional composites textbooks use 3 Young's moduli, 3 Poisson's ratios, and 3 shear moduli. These properties lead to the conclusion that dilatation cannot be separated from distortion in orthotropic materials, a conclusion that fundamentally conflicts with strain induced orthotropy.

A relatively recent development in composite simulation is micromechanics simulations to define macroscopic material properties. Software codes like Digimat or MultiMechanics attempt to model small scale interactions between matrix and reinforcement materials. These approaches are computationally time consuming and are terrible at tracking viscoelasticity & damage. The simulation system 10 offers a continuum level orthotropic solution with viscoelastic damage accumulation. Moreover, failure can happen in distortion or dilatation.

For plasticity, the famous von Mises failure criterion is derived from maximum distortion. The simulation system 10 independently tracks 6 shear relationships instead.

A bi-stable energy state function for bulk can be used to trigger a necking instability in polymers. In dilatational tension, the proposed energy curve would have a second local minimum.

Spectral approach normally used for viscoelasticity (FIG. 5) could also be applied to plasticity. Multiple parallel mechanisms can cause failure. These parallel mechanisms do not necessarily need to be viscoelastic. The energy-strain response of each mechanism would be inspired by materials science. For example, carbide formation at some dilatational strain could change the contribution of that carbide to the overall load response. As another example, parallel mechanisms could describe cracks growing in partially stabilized zirconia. Current state of the art for plasticity does not consider 6 independent shear relationships, each of which can potentially be affected by dilatation.

For fluid mechanics, such as that shown in FIG. 5 without the 2 springs on the left, shear thinning fluids can be simulated in constant strain rate loading, plateau stress decreases, because Maxwell element specific Gibbs free energy sub state increases, accelerating time and, lowering reduced time strain rate. For Bingham fluids, weak cross-link network similar to Mullins in rubber must be overcome before flow can begin. Unlike rubber, there are no chemically bonded crosslinks to prevent flow.

For Tribology, the simulation system 10 incorporates hydrostatic pressure into viscoelastic fluid behavior. For shear thickening fluids, softening from Gibbs Free Energy is less of an effect compared to stiffening from increased real time strain rate.

Viscosity is the primary material property in fluids, as compared to modulus (i.e., stiffness) used in solids. Viscosity changes with strain rate, but it does not change with time at a given strain rate. In other words, viscosity is not a functional strain history, like viscoelastic modulus. This complicates viscoelastic fluid constitutive modeling.

Classical fluid mechanics consider shear viscosity and stretch viscosity. The simulation system 10 considers 6 shears. Moreover, liquid fluid mechanics is classically considered incompressible, ignoring the viscoelastic bulk response.

Finally, non-Newtonian fluids are separated into different categories with their own constitutive laws. These include shear thinning, shear thickening, and Bingham fluids. The simulation system 10 unifies all of these into a single theory.

For molecular dynamics scale-up, the simulation system 10 uses MD simulations to guide shape of energy functions. FIGS. 3 and 4 show points from molecular dynamics simulations. This work was done with Schroedinger, but the simulation system 10 integrates these results into a continuum-level model. MD is limited by extremely short time scales. The viscoelastic simulation disclosed herein can compensate for such short times by simulating short time stress relaxation tests at elevated temperature, since temperature accelerates time. In this way, the simulation system 10 predicts time & temperature response of simulated polymers.

MD simulations enable virtual chemistry, allowing materials companies to iterate quickly through many iterations and alleviate safety concerns from generating unknown compounds. Unfortunately, MD mechanical simulations require massive computation time, for example a microsecond event on a 40 nanometer polymer cube can take days to simulate. Scaling up to the continuum level is considered the “Holy Grail” for the industry. The simulation system 10 provides for such a scale up.

For foam, localized buckling on the microstructure length scale complicates foam simulation. The nonlinear springs in the generalized Maxell model (shown in FIG. 5) can be tailored to include the initial local peak and the plateau that results from the microstructure buckling instability. The mechanical response of closed cell foams can be dominated by the pneumatic contribution. The bulk contribution to stress can include an isotropic pneumatic spring, whose response is based on the ideal gas law. The mechanical response of closed cell foams can be dominated by the pneumatic contribution. The bulk contribution to stress can include an isotropic pneumatic spring, whose response is based on the ideal gas law. When modeling foam, viscoelasticity and closed cell response is commonly ignored or at least avoided. The simulation system 10 makes these problems manageable.

For multi-body simulation, the simulation system 10 can be combined with finite element analysis to build a library of components for multi-body simulation, enabling time & temperature dependence in rubber bushings and tires. The limitations of rubber bushings & tires are a well-known problem for multi-body dynamic simulation software. Some libraries exist, but they do not typically consider frequency or temperature dependence. The simulation system 10 can be used in conjunction with Finite Element Analysis (FEA) to build proper nonlinear viscoelastic libraries.

The simulation system 10 simulates material properties and failure criteria. Simulation is a powerful tool for product development, because products and their sub-systems can be tested virtually. Virtual prototyping enables far more design iterations in a much smaller amount of time and at a substantially lower cost. Simulation accelerates time to market, increases quality and reduces development costs.

Engineering simulation is built on three fundamental pillars: 1) defining the geometry, 2) applying boundary conditions, and 3) defining the material constitutive laws. The simulation system 10 revolves around pillar 3. The simulation system 10 is applicable for finite element analysis, (commonly applied to solids), the finite difference method (commonly used for fluids) and, other solution methods.

Consider the specific example of Abaqus finite element software, where the simulation system 10 is a user defined material model, called a UMAT. The UMAT calculates stresses from strains coming from Abaqus. It also calculates the Jacobian, which is needed by Abaqus' nonlinear solver. The simulation system 10 implements the new mechanics internally, receiving strains and returning stresses in the traditional 6 element tensor format. The simulation system 10 is the material definition, including failure criteria.

Solid mechanics is built with a 6 element stress tensor and a 6 element strain tensor. In finite element analysis, the solver concurrently solves all six for each finite element in the model. The simulation system 10 implements 9 mathematical relationships. Not coincidentally, classical mechanics requires 9 independent material properties for orthotropic materials, which have unique properties in all 3 orthogonal directions. The simulation system 10 relates each shear strain to only one shear stress by one relationship, which speeds calculation speed.

The simulation system 10 is not the first to relate energy to mechanics. Notably, hyperelastic rubber models such as Gent or Arruda-Boyce use what is termed a strain energy density function. Strain Energy Density should have been called volume specific Gibbs free energy change. Nonetheless, energy has previously been defined in terms of strain energy invariants. The simulation system 10 separates free energy into 6 independent shears and one bulk. As such, there are 9 separate relationships relating stress to strain, similar to the 9 material properties in linear elastic orthotropy.

For nonlinear viscoelasticity, a number of polymer constitutive models exist. The mechanics tend to be built on strain invariants rather than 9 strains of the simulation system 10. Many of these viscoelastic models assume incompressibility and are incapable of capturing strain induced anisotropy. All cover some narrow range of temperature and loading history conditions, but none describe the complete response particularly well. Some inferior, competitive constitutive models include Bergstrom-Boyce, Parallel Rheological Framework, Potential Energy Clock, and Free Volume.

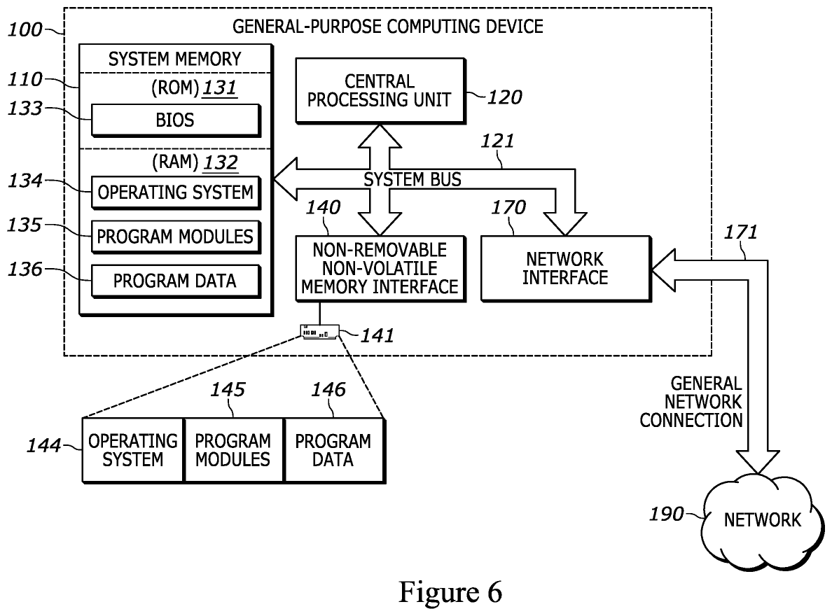

With reference to FIG. 6, an apparatus is disclosed, such as an exemplary general-purpose computing device, for performing the simulation described herein by the. This general-purpose computing device is illustrated in the form of an exemplary general-purpose computing device 100. The general-purpose computing device 100 may be of the type utilized for the control module 20 (FIG. 2). As such, it will be described with the understanding that variations can be made thereto. The exemplary general-purpose computing device 100 can include, but is not limited to, one or more central processing units (CPUs) 120, a system memory 130 and a system bus 121 that couples various system components including the system memory to the central processing unit 120. The system bus 121 may be any of several types of bus structures including a memory bus or memory controller, a peripheral bus, and a local bus using any of a variety of bus architectures. Depending on the specific physical implementation, one or more of the CPUs 120, the system memory 130 and other components of the general-purpose computing device 100 can be physically co-located, such as on a single chip. In such a case, some or all of the system bus 121 can be nothing more than communicational pathways within a single chip structure and its illustration in FIG. 6 can be nothing more than notational convenience for the purpose of illustration.

The general-purpose computing device 100 also typically includes computer readable media, which can include any available media that can be accessed by computing device 100. By way of example, and not limitation, computer readable media may comprise computer storage media and communication media. Computer storage media includes media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, cloud data storage resources, video cards, or any other medium which can be used to store the desired information and which can be accessed by the general-purpose computing device 100. Communication media typically embodies computer readable instructions, data structures, program modules or other data in a modulated data signal such as a carrier wave or other transport mechanism and includes any information delivery media. By way of example, and not limitation, communication media includes wired media such as a wired network or direct-wired connection, and wireless media such as acoustic, RF, infrared and other wireless media. Combinations of the any of the above should also be included within the scope of computer readable media.

When using communication media, the general-purpose computing device 100 may operate in a networked environment via logical connections to one or more remote computers. The logical connection depicted in FIG. 6 is a general network connection 171 to the network 190, which can be a local area network (LAN), a wide area network (WAN) such as the Internet, or other networks. The computing device 100 is connected to the general network connection 171 through a network interface or adapter 170 that is, in turn, connected to the system bus 121. In a networked environment, program modules depicted relative to the general-purpose computing device 100, or portions or peripherals thereof, may be stored in the memory of one or more other computing devices that are communicatively coupled to the general-purpose computing device 100 through the general network connection 171. It will be appreciated that the network connections shown are exemplary and other means of establishing a communications link between computing devices may be used.

The general-purpose computing device 100 may also include other removable/non-removable, volatile/nonvolatile computer storage media. By way of example only, FIG. 6 illustrates a hard disk drive 141 that reads from or writes to non-removable, nonvolatile media. Other removable/non-removable, volatile/nonvolatile computer storage media that can be used with the exemplary computing device include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140.

The drives and their associated computer storage media discussed above and illustrated in FIG. 6, provide storage of computer readable instructions, data structures, program modules and other data for the general-purpose computing device 100. In FIG. 6, for example, hard disk drive 141 is illustrated as storing operating system 144, other program modules 145, and program data 146. Note that these components can either be the same as or different from operating system 134, other program modules 135 and program data 136. Operating system 144, other program modules 145 and program data 146 are given different numbers here to illustrate that, at a minimum, they are different copies.

The embodiments discussed above include a hyperbolic secant function, with angles being used to define distortional strain. In accordance with at least one other embodiment, at least one embodiment discussed below defines distortional strain as the natural log of the ratio of the stretches. This new distortional strain definition eliminates the hyperbolic secant, clarifying the strain definition. In accordance with the embodiments disclosed herein, at least nine (9) independent mathematical relationships are utilized to define their energy function. Typical functions separate dilatation and distortion, but they use the J2 strain invariant for distortion and the first principal strain invariant (I1) to define energy. Thus, typical functions utilize only two mathematical parameters, where the embodiment(s) disclosed herein simulate orthotropy based on at least nine independent mathematical relationships. In the case of strain induced orthotropy, energy of simulated isotropic materials is defined in the principal strain directions. This means three (3) of the mathematical relationships are the three (3) principal directions. Three distortions are then defined in those directions. The 3 dilitational z-function are also defined with the 3 principal strains. Otherwise energy varies with choice of reference directions.

In accordance with at least one other embodiment that builds on the embodiments disclosed above, the simulation system 10 utilizes new mechanics that include a new strain definition for an energy function is disclosed. This new strain definition first defines a new strain(s), then defines an energy function in terms of those strain(s), and thereafter calculates stresses as a derivative of energy. Advantages of such new mechanics include that it separates dilatation and distortion, even for orthotropy, e.g., strain inducted orthotropy, it ties thermodynamics and mechanics together, and provides a foundation to solve complex problems, such as nonlinear viscoelasticity, mixed-mode fracture, viscoelastic damage in composites, rubber mechanics, plasticity, tribology, etc.

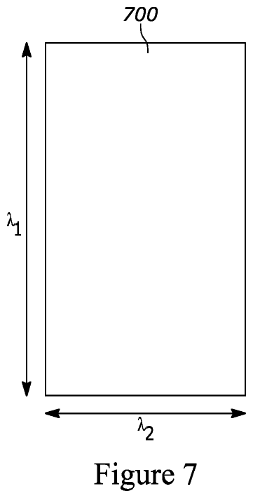

With reference to FIG. 7, the simulation system 10 simulates a material sample 700 that is subject to stretches λ₁ and λ₂ in perpendicular directions along the simulated material. The simulation system 10 calculates, for a first face, pure shear as a log of a ratio of the stretches in accordance with the following equation:

$${\tilde{γ}}_{3} ≡ {ɛ}_{1} - {ɛ}_{2} = ln ( \frac{{λ}_{1}}{{λ}_{2}} )$$

where \tilde{γ}₃ equals pure shear at small strains, and where ε₁ and ε₂ are the true strains in principal directions.

And, the simulation system 10 calculates pure shear at small strains more generally for remaining two faces with the following equation:

$${\tilde{γ}}_{i} ≡ {ɛ}_{i + 1} - {ɛ}_{i - 1} = ln ( \frac{{λ}_{i + 1}}{{λ}_{i - 1}} ) ,$$

where i=1,2,3, 1, 2.

The simulation system 10 defines dilatational energy in terms of principal true strains, as visualized in FIG. 2C. The simulation system 10 calculates isotropic small strain with the following formula:

Δg_{b}=½κ(ε₁+ε₂+ε₃)²

The simulation system 10 further calculates large strain with the following equation:

Δg_{b}=½κ{z₁(ε₁)+z₂(ε₂)+z₃(ε₃)}²,

where z's are dilatation functions and epsilons are in the principal strain directions. For simulation of stress and strain for an orthotropic composite material discussed in more detail below, the epsilons within this equation are the strains in the principal directions of orthotropy, kappa is bulk modulus and the z functions combine into the dilatational contribution to free energy.

Thus, dilatational energy defined as sum of three z functions, where each z function depends on only one orthogonal strain. For example, with equitriaxial loading on an anisotropic cube, different stresses are needed in 3 orthogonal directions. A derivative of bulk energy provides these unique stresses.

The simulation system 10 further defines strain energy density in principal directions for initially isotropic materials according to the following equation:

Δgₘ=Δg_{B}(ε₁,ε₂,ε₃)+⅔bΣᵢ₌₁³Δgᵢ(\tilde{γ}ᵢ)

where b is the vertical shift factor that is a function of dilatation, linking dilatation and distortion.

The system 10 then calculates stresses from energy according to the following equation:

$${σ}_{i j} = \frac{\partial Δ {g}_{m}}{\partial {ɛ}_{i j}}$$

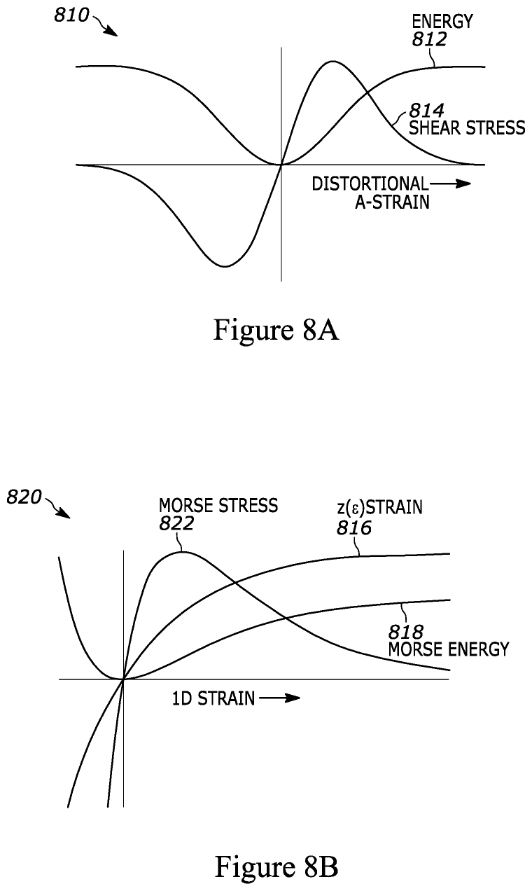

With reference to FIGS. 8A and 8B, examples shapes of energy functions are illustrated as including at least nine (9) relationships for strain induced orthotropy, as calculated by the simulation system 10. In particular, FIG. 8A illustrates a first graph 810 including distortional A-strain as the x-axis and two curves, an energy curve 812 and a shear stress curve 814. FIG. 8B illustrates a second graph 820 including 1D strain as the x-axis and three curves, a z(ε) strain curve 816, a Morse energy curve 818, and a Morse stress curve 822.

The simulation system 10 calculates stresses in principal directions based on calculated distortional stress according to the following equations:

For example, the simulation system 10 can predict, for an isotropic sample in uniaxial loading, linear elasticity, plasticity, and fracture if bulk and shear energy functions are known using the following equations:

Thus, as shown with these equations dilatation must balance distortion in transverse directions.

In accordance with at least one other embodiment that builds on the embodiments disclosed above, the simulation system 10 utilizes new mechanics that include a new strain definition, energy and composites. For simulated materials that are already orthotropic, the simulation system 10 continues to use 6 shear and 3 bulk strains. Similarly as discussed above, this new strain definition first defines a new strain(s), then defines an energy function in terms of those strain(s), and thereafter calculates stresses as a derivative of energy. Advantages of such new mechanics include that it completely separates dilatation and distortion, even for orthotropy, that it ties thermodynamics and mechanics, and provides for a foundation to solve complex problems, such as MD scaleup, viscoelastic damage accumulation, cavitation failure, thermoplastic self-healing, polymer processing and thermoplastic flow, and Environmental effects, e.g., temperature, solvent.

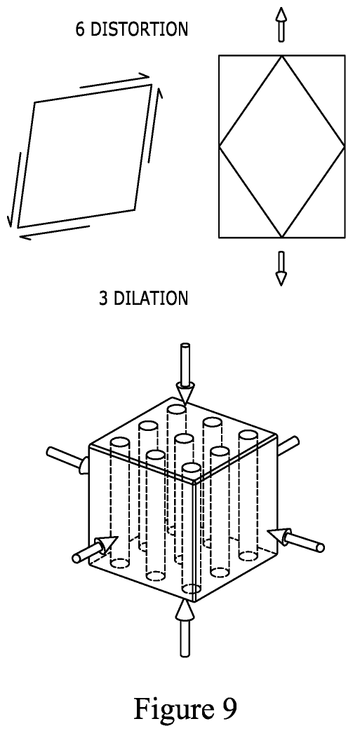

With reference to FIG. 9, the simulation system 10 calculates distortion in 6 axis and dilation in 3 axis, as shown. For simulated orthotropic materials, the simulation system 10 calculates small strain based on nine (9) independent material properties and large strain based on nine (9) unique stress-strain relationships valid to large strains. The simulation system 10 calculates energy that transforms the 9 unique relationships into six (6) element stress tensor according to the following equation:

$$\frac{\partial Δ {g}_{m}}{\partial {ɛ}_{i}} = {σ}_{i}$$

Simulated orthotropic materials are subject to the stretches λ₁ and λ₂ as shown in FIG. 7. The simulation system 10 calculates pure shear for simulated orthotropic materials as a log of a ratio of the stretches, as discussed above. The simulation system 10 further defines dilatational energy for simulated orthotropic materials in terms of principal true strains, as discussed above. The simulation system 10 further defines strain energy density for simulated orthotropic materials in principle directions according to the following equation:

Δgₘ=Δg_{B}(ε₁,ε₂,ε₃)+⅔bΣᵢ₌₁³Δgᵢ(\tilde{γ}ᵢ)+bΣᵢ₌₄⁶Δgᵢ(\tilde{γ}ᵢ)

where b is the vertical shift factor as a function of dilatation, linking dilatation, and distortion.

The simulation system 10 further calculates for simulated orthotropic materials stresses from energy according to the following equation (as discussed above):

$${σ}_{i j} = \frac{\partial Δ {g}_{m}}{\partial {ɛ}_{i j}}$$

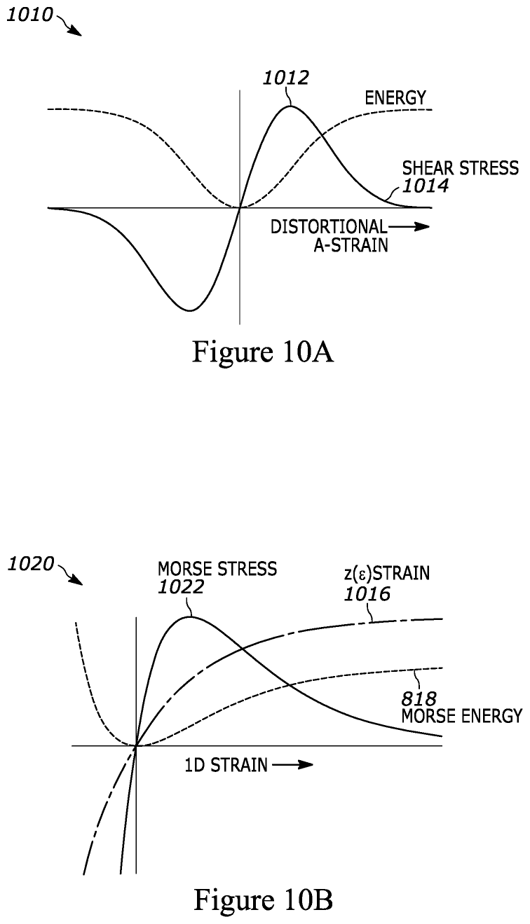

With reference to FIGS. 10A and 10B, examples shapes of energy functions for simulated orthotropic materials are illustrated as including at least nine (9) relationships for strain induced orthotropy, six (6) distortional strains and three (3) dilatational strains, as calculated by the simulation system 10. In particular, FIG. 10A illustrates a first graph 810 including distortional A-strain as the x-axis and two curves, an energy curve 1012 and a shear stress curve 1014. FIG. 10B illustrates a second graph 1020 including 1D strain as the x-axis and three curves, a z(ε) strain curve 1016, a Morse energy curve 1018, and a Morse stress curve 1022.

The simulation system 10 calculates stresses for simulated orthotropic materials in principal directions based on calculated distortional stress, as discussed above. The simulation system 10 further can predict, for an isotropic sample for simulated orthotropic materials in uniaxial loading, linear elasticity, plasticity, and fracture if bulk and shear energy functions, using the equations disclosed above.

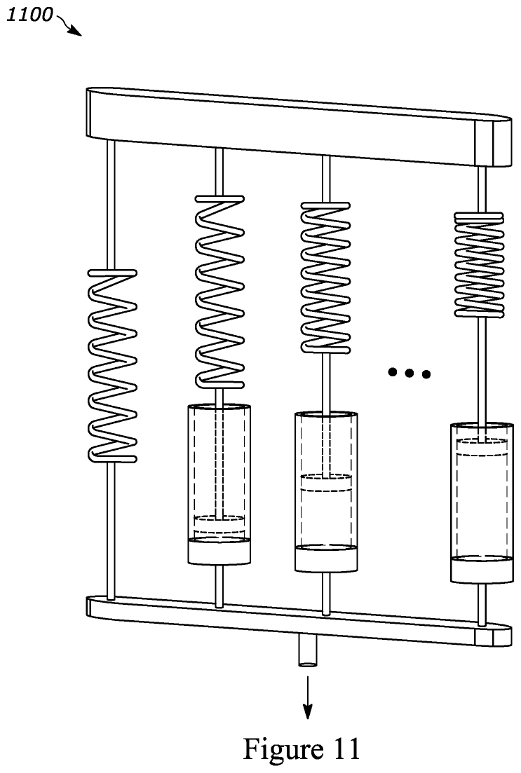

With reference to FIG. 11, a Maxwell model 1100 for simulated orthotropic and non-orthotropic materials is shown having nonlinear viscoelasticity, as utilized by the simulation system 10. The Maxwell model 1100 calculates entropic elasticity with a crosslink network. The Maxwell model 1100 includes nonlinear springs that store energy as changes in Gibbs free energy, with stress being derivative according to the following equation:

$$\frac{\partial Δ {g}_{m}}{\partial {ɛ}_{i}} = {σ}_{i}$$

The Maxwell model 1100 includes a nonlinear viscoelastic (NLVE) response Eyring Polanyi reduced time according to the following equation:

$$a = \frac{T}{{T}_{R}} {e}^{- \frac{Δ {g}^{†}}{R T}}$$

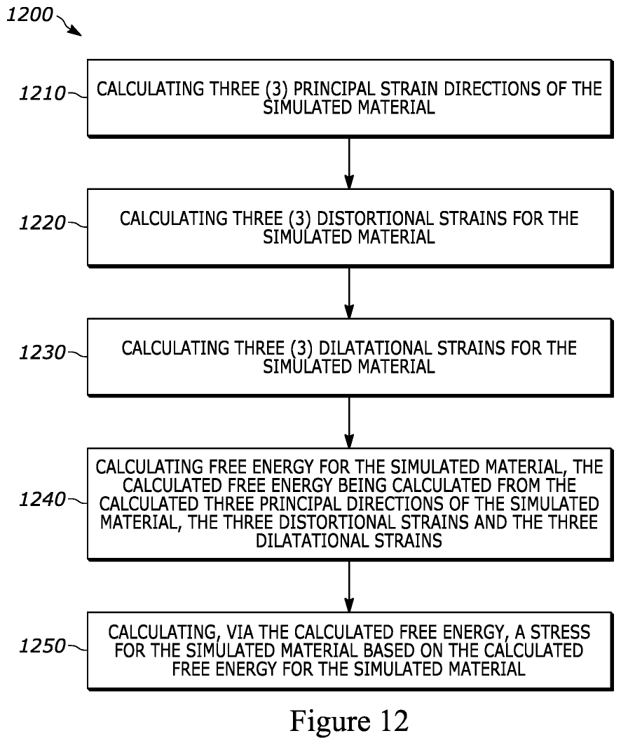

FIG. 12 illustrates a method 1200 for simulating strain induced orthotropy for a material. The method 1200 is stored on a non-transitory storage medium (e.g., ROM 131 and/or hard disk drive 141) and executed by a processor, such as the CPU 120.

The method 1200 includes a process 1210 calculating three (3) principal strain directions of the simulated material. Process 1210 proceeds to process 1220.

Process 1220 includes calculating three (3) distortional strains for the simulated material. Process 1220 proceeds to process 1230.

Process 1230 includes calculating three (3) dilatational strains for the simulated material. Process 1230 proceeds to process 1240.

Process 1240 includes calculating free energy for the simulated material, the calculated free energy being calculated from the calculated three principal directions of the simulated material, the three distortional strains and the three dilatational strains. Process 1240 proceeds to process 1250

Process 1250 includes calculating, via the calculated free energy, a stress for the simulated material based on the calculated free energy for the simulated material.

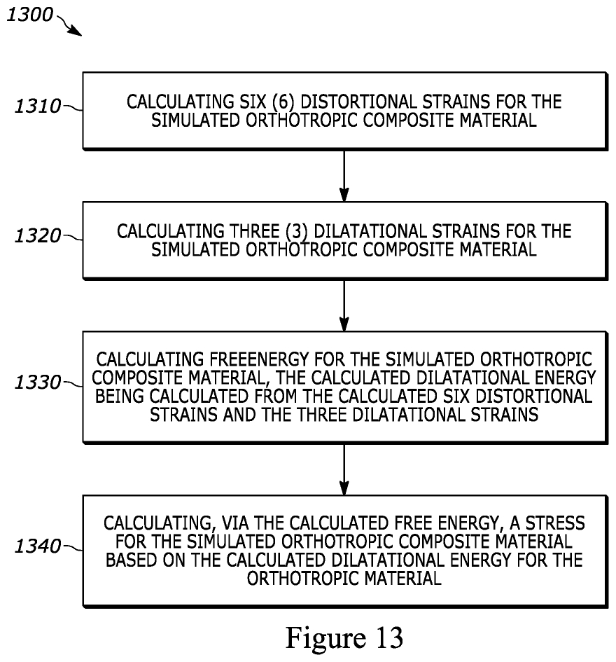

FIG. 13 illustrates another method 1300 for simulating stress and strain for an orthotropic composite material. The method 1300 is stored on a non-transitory storage medium (e.g., ROM 131 and/or hard disk drive 141) and executed by a processor, such as the CPU 120.

The method 1300 includes a process 1310 calculating six (6) distortional strains for the simulated orthotropic composite material. Process 1310 proceeds to process 1320.

Process 1320 includes calculating three (3) dilatational strains for the simulated orthotropic composite material. Process 1320 proceeds to process 1330.

Process 1330 includes calculating free energy for the simulated orthotropic composite material, the calculated dilatational energy being calculated from the calculated six distortional strains and the three dilatational strains. Process 1330 proceeds to process 1340.

Process 1340 includes calculating, via the calculated free energy, a stress for the simulated orthotropic composite material based on the calculated dilatational energy for the orthotropic material.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

What is claimed is:

1. A method, stored on a non-transitory medium and executed by a processor, for simulating strain induced orthotropy for a material, the method comprising:
calculating three (3) principal strain directions of the simulated material;
calculating three (3) distortional strains for the simulated material;
calculating three (3) dilatational strains for the simulated material;
calculating free energy for the simulated material, the calculated free energy being calculated from the calculated three principal directions of the simulated material, the three distortional strains and the three dilatational strains; and
calculating, via the calculated free energy, a stress for the simulated material based on the calculated free energy for the simulated material.

2. The method according to claim 1, wherein the dilatational energy is defined in terms of large strain according to the following equation:
Δg_{b}=½κ{z₁(ε₁)+z₂(ε₂)+z₃(ε₃)}²,
where z's are dilatation functions and epsilons are in the principal strain directions.

3. The method according to claim 1, further comprising:
defining the distortional strains for a face as a log of a ratio of stretches of the simulated material according to the following equation:
$${\tilde{γ}}_{3} ≡ {ɛ}_{1} - {ɛ}_{2} = ln ( \frac{{λ}_{1}}{{λ}_{2}} ) ,$$
where \tilde{γ}₃ equals pure shear at small strains, ε₁ and ε₂ are the true strains in principal directions, λ₁ and λ₁ are stretches in perpendicular directions along the simulated material; and
defining the distortional strains for remain faces as a log of a ratio of stretches of the simulated material according to the following equation:
$${\tilde{γ}}_{i} ≡ {ɛ}_{i + 1} - {ɛ}_{i - 1} = ln ( \frac{{λ}_{i + 1}}{{λ}_{i - 1}} ) ,$$
where i=1, 2, 3, 1, 2.

4. The method according to claim 1, wherein the calculated stress is calculated in principal orthotropic directions according to the following equations:

[symbol]

5. The method according to claim 1, wherein further comprising calculating entropic elasticity with a crosslink network in parallel to a generalized Maxell model, the Maxwell elements including nonlinear springs that store energy as volume specific Gibbs free energy, with stress being derived according to the following equation:
$$\frac{\partial Δ {g}_{m}}{\partial {ɛ}_{i}} = {σ}_{i} .$$

6. A method, stored on a non-transitory medium and executed by a processor, for simulating stress and strain for an orthotropic composite material, the method comprising:
calculating six (6) distortional strains for the simulated orthotropic composite material; and
calculating three (3) dilatational strains for the simulated orthotropic composite material;
calculating free energy for the simulated orthotropic composite material, the calculated dilatational energy being calculated from the calculated six distortional strains and the three dilatational strains; and
calculating, via the calculated free energy, a stress for the simulated orthotropic composite material based on the calculated dilatational energy for the orthotropic material.

7. The method according to claim 6, wherein the dilatational energy is defined in terms of large strain according to the following equation:
Δg_{b}=½κ{z₁(ε₁)+z₂(ε₂)+z₃(ε₃)}²,
where epsilons are the strains in the principal directions of orthotropy, kappa is bulk modulus and the z functions combine into the dilatational contribution to free energy.

8. The method according to claim 6, wherein the distortional strains are defined by an angle, which leads to a hyperbolic secant function in the stress tensor calculation.

9. The method according to claim 6, further comprising defining the distortional strains as a log of a ratio of stretches of the simulated material according to the following equation:
$${\tilde{γ}}_{3} ≡ {ɛ}_{1} - {ɛ}_{2} = ln ( \frac{{λ}_{1}}{{λ}_{2}} ) ,$$
where \tilde{γ}₃ equals pure shear at small strains, ε₁ and ε₂ are the true strains in principal directions, and are stretches in perpendicular directions along the simulated material; and
defining the distortional strains for the remain faces as a log of a ratio of stretches of the simulated material according to the following equation:
$${\tilde{γ}}_{i} ≡ {ɛ}_{i + 1} - {ɛ}_{i - 1} = ln ( \frac{{λ}_{i + 1}}{{λ}_{i - 1}} ) ,$$
where i=1, 2, 3, 1, 2.

10. The method according to claim 6, further comprising calculating entropic elasticity with a crosslink network in parallel to a generalized Maxell model, the Maxwell elements including nonlinear springs that store energy as volume specific Gibbs free energy, with stress being derived according to the following equation:
$$\frac{\partial Δ {g}_{m}}{\partial {ɛ}_{i}} = {σ}_{i} .$$

11. The method according to claim 6, wherein the calculated stress is calculated in principal orthotropic directions according to the following equations: [symbol]
